(19) United States
(12) Patent Application Publication
(10) Pub. No.: US 2003/0008397 A1
(43) Pub. Date: Jan. 9, 2003

(54) Coupled peptides

(76) Inventors: Gerrit Jan Beumer, Heteren (NL); Ronald Christopher Chatelier, San Diego, CA (US); Hans Jorg Griesser, The Patch (AU); Graham Johnson, Peakhurst (AU); Keith Michael McLean, Mentone (AU)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/197,036

(22) Filed: Jul. 17, 2002

Related U.S. Application Data
(63) Continuation of application No. 09/438,684, filed on Nov. 12, 1999.

(30) Foreign Application Priority Data
Nov. 13, 1998 (DE) 98121663.3

(51) Int. Cl. C12N 5/02; B05D 3/00

(52) U.S. Cl. 435/402; 427/2.11; 427/2.24

(57) ABSTRACT

The present invention is directed to a process for coupling an adhesive glycoprotein to a surface of a bulk material comprising the following steps:

a) a layer of a volatile aldehyde is deposited to the surface of the bulk material from a gas plasma atmosphere,

b) the layer so formed on the bulk material is contacted with a cell-adhesive glycoprotein having amino groups,

c) the coupling of the amino groups of the glycoprotein to the carbonyl groups of the deposited aldehyde layer is strenghthened by transforming the primarily formed —CH═N— bonds in the presence of a reducing agent into —CH₂—NH— bonds;

and to a cell growth material comprising a bulk material having at least one surface to which a cell adhesive glycoprotein having amino groups is coupled characterized in that said amino groups are covalently bonded to aldehyde groups of a layer of a volatile aldehyde, deposited to the surface of said bulk material from a gas plasma atmosphere, via first formed —CH═N— groups which have been transformed into —CH₂—NH— groups in the presence of a reducing agent.

The present invention is directed to a cell growth material comprising a bulk material to the surface of which an adhesive glycoprotein is covalently coupled, and to a process of covalently coupling an adhesive glycoprotein to the surface of a bulk material.

The invention provides materials, and a method for fabricating such materials, that promote colonization by anchorage-dependent cells. This invention provides new materials for in vitro cell culture but is particularly advantageous for applications that require tight apposition in vivo between a biomedical device and biological (particularly mammalian) tissue. Thus, the new materials are particularly intended for biomedical devices with improved biocompatibility, especially ophthalmic devices. Examples are a corneal onlay and a keratoprosthesis, both of which require close integration with ocular tissue and re-attachment and growth of epithelial cell layers.

Synthetic materials generally have insufficient affinity to biological environments (cells in culture, medical implants, etc.). The invention describes a method to overcome this limitation and thus enables use of materials that possess suitable “bulk” properties (such as mechanical, optical, flexibility, biostability, etc.) but do not by themselves promote the required rapid and effective colonization by anchorage-dependent cells. The coatings of the present invention achieve rapid in vitro cell attachment from cell suspensions onto a cell growth material or a biomedical device, and effective in vivo tissue attachment. The coatings also enable rapid colonization of a biomedical device by conferring the ability of cell layers to grow from the rims onto the coated surface(s). The coatings furthermore enhance integration of biomedical devices when they are placed in contact with a soft tissue environment.

The adsorption of cell-adhesive glycoproteins onto bulk materials to promote cell colonization is well known for in vitro applications such as cell culture. However, when such adsorbed protein layers are placed in contact with biological media, other proteins, and possibly lipids, from the biological medium can discplace the initially adsorbed adhesive glycoproteins from the surface of the bulk material. As a result, the biological/material interface becomes less well defined over time, the adsorbed protein layer is ill-defined and uncontrollable, and control is lost over the host response. This is expected to be particularly so and rapid when fluid dynamic motions lead to more rapid desorption/adsoprtion exchange of surface-adsorbed biomolecules. For instance, the anterior surface of a corneal inlay or a keratoprosthesis needs to be colonized by migration from the implant rim of epithelial cells. Such migration is markedly enhanced by the presence of adhesive glycoproteins on the device. Howeve, the blinking motion of the eyelid causes tangential fluid flow with high turbulence over the device surface. Thus, surface-adsorbed molecules are subject not only to diffusional exchange but also to additional desorption forces by the rapid stirring of the aqueous boundary layer.

It is thus beneficial to covalently immobilize cell-adhesive glycoproteins onto device surfaces in order to prevent their removal/displacement by exchange with other proteins and lipids. A number of attachment methods are known in the art. However, drawbacks exist. For instance, glutaraldehyde fixation leads to crosslinking within and between glycoproteins, and this causes changes to secondary and tertiary structures. It is known that a large fraction of proteins thus immmobilized are not longer biologically active. A method for the mild covalent immobilization of adhesive glycoprotein is thus required that leads to minimal structure changes and maximal effectiveness of the immobilized protein layer.

The present invention meets these needs. It provides a novel and highly effective method for surface-immobilizing cell-adhesive glycoproteins, and composite materials (bulk material plus thin layer coatings) that are highly effective in cell-contacting applications. Results obtained illustrate the high effectiveness of the glycoprotein layers produced by the method of the current invention for promoting epithelial cell colonization.

In more detail, the invention describes materials that are capable of enabling rapid and effective attachment and growth of mammalian, anchorage-dependent cells on their surfaces by virtue of the presence of an immobilized layer of cell-adhesive glycoproteins on one or multiple surfaces. The thin layer of adhesive glycoprotein is covalently immobilized on to the surface of a bulk material using a thin interfacial bonding layer deposited from a gas plasma (glow discharge) atmoshpere comprising an aldehyde compound. Thus, the materials of the present invention comprise, schematically, a composite structure of three layers. The first layer is the bulk material. The second layer is an aldehyde-containing interfacial bonding layer deposited from a gas plasma. The third layer, which contacts the cells, comprises an adhesive glycoprotein.

The bulk material can be e.g. a synthetic polymer, a natural polymer, a ceramic, or a metallic material. Examples for commercial available bulk materials are e.g. membranes such as poretics membranes, or Teflon (FEP) membranes. In general, polymeric materials are preferred bulk materials, for example those polymeric materials which have been disclosed in WO 96/31546, WO 96/31545, WO 96/31547, or WO 97/00274. Furthermore, polymeric materials comprising perfluoropolyether segments, or siloxane segments, or both in combination, are suitable bulk materials. It may also be advantageous if said materials are porous, for example as disclosed in WO 97/35904, WO 97/35905, WO 97/35906, or, in more general terms, in WO 95/13764.

With respect to the third layer various cell-adhesive glycoproteins are known in the art: collagens (various types), fibronectin, vitronectin, laminin, and the like. The present invention is applicable to any adhesive glycoprotein that contains amino groups, preferably that contains lysine residues.

It is also within the scope of this invention that said third layer comprises in addition to an adhesive glycoprotein one or more other biologically active molecules. Such molecules can be for instance other proteins, glycosaminoglycans, or polysaccharides. According to the invention, such molecules can be co-immobilised with adhesive glycoproteins in order to produce implantable materials. Naturally, it would be necessary for these other units to be carefully chosen on the basis of their biological signalling properties which would make them suitable for use in the particular application concerned. However, in one embodiment of the invention it is preferred to exclude peptoids from the molecules forming the third layer. In a further embodiment it is preferred that the third layer consists essentially of adhesive glycoprotein, or more pronounced, consists only of adhesive glycoprotein.

The interfacial bonding layer, the “aldehyde plasma polymer”, is deposited from a gas plasma which contains a volatile aldehyde compound and optionally other constituents, for example a carrier gas such as argon. The aldehyde plasma polymer layer functions both as an interfacial bonding layer and as a surface activation step for bulk materials whose surfaces do not inherently possess chemical groups capable of undergoing chemical reaction with cell-adhesive glycoproteins. Fabrication of the interfacial bonding layer by plasma deposition confers a unique advantage in that such a plasma coating adheres exceptionally stronlgy to most bulk materials, and can be readily deposited onto most bulk materials of biomedical interest, thus allowing ready transferability of this invention to a range of bulk materials. The interfacial bonding layer containing aldehyde groups also confers a unique advantage in that it enables immobilization of adhesive glycoproteins under mild aqueous reaction conditions onto bulk biomaterials that otherwise would allow protein attachment only under much harsher chemical conditions.

In general, the aldehyde plasma deposition may occur in a manner known per se. However, while commonly the deposition of plasma polymer coatings is executed at reduced pressure, typically in the range 0.1 to 1 Torr, aldehyde plasma polymer layers suitable for the present invention can also be deposited at atmospheric pressure using suitable equipment.

A preferred volatile aldehyde has up to 9 carbon atoms, preferably 2 to 7 carbon atoms, more preferred up to 4 carbon atoms, and even more preferred 2 to 4 carbon atoms. The most preferred aldehyde is acetaldehyde, while propionaldehyde can still be recommended. The volatile aldehydes as taught in this invention for the deposition of the interfacial bonding layer provide much better quality films, in terms of cohesion, than the use of formaldehyde aqueous solution as disclosed in the prior art. It is a consequence, therefore, of the present invention that it is preferred to conduct the plasma step of the process according to this invention (see step a) of claim 1) in the absence, or substantial absence of water.

The interfacial immobilization reaction between the surface of the aldehyde plasma polymer and the adhesive glycoprotein causes formation of interfacial Schiff base bonds. While the invention includes formation of Schiff base linkages (—CH═N—) a preferred embodiment is that these Schiff base linkages are subsequently treated by reductive amination, preferably by treatment with a reducing agent, e.g. cyanoborohydride. Such reductive amination improves the strength of covalent immobilization.

In view thereof, one embodiment of this invention is a process for coupling an adhesive glycoprotein to a surface of a bulk material comprising the following steps:

a) a layer of a volatile aldehyde is deposited to the surface of the bulk material from a gas plasma atmosphere,

b) the layer so formed on the bulk material is contacted with a cell-adhesive glycoprotein having amino groups,

c) the coupling of the amino groups of the glycoprotein to the carbonyl groups of the deposited aldehyde layer is strenghthened by transforming the primarily formed —CH═N— bonds in the presence of a reducing agent into —CH₂—NH— bonds.

Another embodiment is a cell growth material comprising a bulk material having at least one surface to which a cell adhesive glycoprotein having amino groups is coupled characterized in that said amino groups are covalently bonded to aldehyde groups of a layer of a volatile aidehyde, deposited to the surface of said bulk material from a gas plasma atmosphere, via first formed —CH═N— groups which have been transformed into —CH₂—NH— groups in the presence of a reducing agent.

Preferred elements in the process or material of the invention have been defined hereinbefore. Some of the separate elements of the invention are well known to the person skilled in the art, such as plasma polymer deposition, or transformation of —CH═N— groups into —CH₂—NH— groups. It is therefore believed that the present disclosure is fully enabling even in the absence of lengthy description of technology which is known per se, although such technology, of course, is not known in the context of the invention.

Surprisingly, the adhesive glycoproteins surface-immobilized by the route of the present invention are still highly capable of promoting cell attachment and proliferation. The chemical interfacial reaction that leads to a Schiff base linkage is believed to be nonspecific in the sense of not targeting a particular part of the glycoprotein but, instead, any amino group, or lysine residue, wherever it is located, and of not leading to any particular spatial orientation of the immobilized glycoprotein molecules. Further surprisingly, such putatively random reaction and orientation still enables high effectiveness for attached glycoproteins to present the cell-adhesive epitope to approaching cells. This presents a clear advantage compared with known methods for immobilizing cell-adhesive glycoproteins such as glutaraldehyde-based methods that cause crosslinking within and between glycoprotein molecules and thus lead to changes in the secondary and tertiary protein structure.

The cell growth materials, or implantable bulk materials, of the present invention are many and varied and include the following which are listed here by way of example: wound repair materials, synthetic skin or connective tissue, ocular implants such as implanted contact lenses and synthetic epikeratoplasties or corneal grafts, orthopaedic implants such as prosthetic joints or synthetic arterial surfaces, synthetic tendon or ligament tissues or materials used to secure bone or ligament in surgical procedures, synthetic neural tissue, prosthetic organs such as apparatus which will carry out the function of the heart, lungs, etc, components of blood contacting devices, immunoassays, antigen/antibody detection kits, affinity matrices etc., other synthetic bioactive apparatus such as heart pacemakers or other synthetic implantable materials.

It is to be understood that the examples provided hereinbefore are not intended to limit the scope of the invention in any way, and that the present invention relates to implantable bulk materials of all types which may need to be implanted into a human or animal body, and will require a surface coating of an adhesive glycoprotein according to the invention to initiate cellular attachment.

The invention will now be described further with reference to the following non-limiting examples:

### EXAMPLE 1

### Plasma Polymer Deposition

This is a standard operating procedure for acetaldehyde plasma deposition. In a laboratory-scale plasma deposition equipment, a dry and clean substrate is placed on a 9 cm diameter electrode, either directly or onto a FEP (fluorinated co-ethylene propylene) sheet (which is used as a disposable thin layer to reduce the need for frequently cleaning the electrode). One pumps down to base pressure checking for air leaks. One pre-rinses twice and then fills to ⅔ a round bottom flask with acetaldehyde monomer (Aldrich, 99%, cat#11.007-8). Monomer and flask are outgased for 1-2 minutes at 0.1 Torr, then the monomer feed valve is closed. The reactor is pumped down to base pressure again, the shunt valve is closed, the monomer feed valve is opened. One throttles to a setting of −12-, reads and records stabilized pressure. The plasma is ignited. Timing is started when constant power is reached. The forward power is readjusted during treatment such that a constant load power results, which usually applies about 15 seconds after ignition, when the plasma becomes stabilized. The pressure rise is monitored, and pressures logged at 30 and 60 seconds. The radio frequency power is then switched off. Monomer feed is continued, allowing pressure to come down to 0.300 Torr, which usually takes 2-3 minutes. Then the monomer feed valves are shut. The reactor is shut. This procedure deposits a 10-20 nanometer thick acetaldehyde plasma polymer layer on bulk substrates.

The plasma conditions are as follows: Upper electrode active Power Load 5 Watts; Forward about 35 W; Radio Frequency: 125 Hz; Monomer pressure: 0.30+/−0.005 Torr; Treatment Time: 60 seconds.

### EXAMPLE 2

### Immobilization of Glycoproteins:

This is a standard operating procedure for Collagen I (Vitrogen) immobilisation using reductive amination onto acetaldehyde plasma polymer (AApp) coated bulk material. The Collagen material used is Vitrogen 100, min. 95% bovine collagen type I, Collagen Corp., CA. USA. A 50 microgram/ml collagen solution in phosphate buffered saline (PBS) is prepared at pH 7.4. A freshly deposited AApp/bulk specimen is incubated at 4° C. in collagen solution. Excess sodium cyanoborohydride (SIGMA, MW 62.84, min. 90%, cat#S 8628) is added and incubated overnight at 4° C., then 2 hours at room temperature. The sample is rinsed 2 times and then soaked in PBS. For XPS analysis, soak duplicate sample in MilliQ watter. Since collagen preprations are perishable, sterile autoclaved equipment is used (pipette tips, glassware, solutions) throughout. Further, collagen I type molecules easily form gels at room temperature under non-acidic conditions. Therefore, glassware, solutions etc. are pre-cooled, and one works on ice, using pre-cooled and buffered solutions.

### EXAMPLE 3

### Surface Analysis:

The data measured of a sample of Example 2 confirm that a thin layer of collagen has been covalently immobilized onto the substrate, and that the bond strength is sufficient to resist autoclaving after the reduction step, whereas without reduction a substantial part of the attached collagen is removable.

In the following tables 1 to 4, which provide surface analytical data obtained by X-ray photoelectron spectroscopy (XPS/ESCA) verifying the coating process, FEP means fluorinated co-ethylene propylene, and AApp means acetaldehyde plasma polymer.

TABLE 1

| | % | % | % | % |
|---|---|---|---|---|
| Sample | Carbon | Fluorine | Oxygen | Nitrogen |
| Teflon FEP | 35 | 65 | | |
| FEP + AApp | 78 | 0 | 21.2 | 0.4 |
| FEP + AApp + Collagen I + | 71.5 | 0.3 | 15.3 | 13.2 |
| NaCNBH3 | | | | |
| FEP + AApp + Collagen I + | 72 | 0 | 15.3 | 13.2 |
| NaCNBH3 + Autoclaving | | | | |
| FEP + AApp + Collagen I, no | 68.1 | 0 | 21.2 | 10.3 |
| NaCNBH3 | | | | |
| FEP + AApp + Collagen I, no | 73 | 0 | 19.3 | 7.7 |
| NaCNBH3 + Autoclaving | | | | |
| FEP + Collagen I | 35.6 | 60.6 | 1.8 | 1.9 |
| FEP + Collagen I + | 35.9 | 61.4 | 1.7 | 1.0 |
| Autoclaving | | | | |

Table 1 shows:

the acetaldehyde plasma polymer covers the Teflon substrate uniformly with no gaps, to a thickness exceeding 10 nm (which is the XPS probe depth), by the absence of a fluorine signal in line 2;

by the marked increase in the nitrogen content between lines 2 and 3 that Collagen I can be immobilized effectively onto acetaldehyde plasma polymer. The nitrogen signal corresponds to a close-packed monolayer of collagen, with no significant gaps in the collagen coating;

that the immobilized collagen layer is firmly (i.e. covalently) attached since it is resistant to removal by autoclaving: compare lines 3 and 4;

that attachment without reduction is less effective: line 5 shows that at the Schiff base stage there remains less collagen I bound to the surface after thorough rinsing, compared with when collagen I is attached with reduction, line 3;

lines 5 and 6 show that collagen I, immobilized via a Schiff base linkage, is susceptible to removal of some of the molecules by autoclaving. This means that one would also have to expect some slow losses when the coated sample is stored at room temperature. Hence, Schiff-base-linked collagen I may not be satisfactory for biomedical devices that require an extended shelf life between fabrication and end-use;

line 7 shows that without the plasma-aldehyde interlayer, collagen I binding onto Teflon FEP is very inefficient, with very low coverage (much below monolayer) and line 8 shows that such physisorbed molecules are prone to removal.

TABLE 2

| | % | % | % | % |
|---|---|---|---|---|
| Sample | Carbon | Fluorine | Oxygen | Nitrogen |
| Teflon FEP | 35 | 65 | 0 | 0 |
| FEP + Acrolein pp | 82.7 | 0 | 16.7 | 0.5 |
| FEP + Acrolein pp + | 67.8 | 0 | 18.3 | 13.7 |
| Collagen I + NaCNBH3 | | | | |
| FEP + Acrolein pp + | 66.8 | 0 | 17.5 | 14.7 |
| Collagen I (Grazing | | | | |
| angle) | | | | |

Table 2 shows that on a plasma-deposited acrolein polymer film, collagen I can be immobilized, again to full monolayer coverage (i.e. there are no significant gaps in the collagen coating). Line 4 (grazing angle XPS, which has a reduced probe depth) shows that the collagen I molecules are located at the very surface, i.e. they do not diffuse into the plasma-aldehyde layer where their biological function could be rendered ineffective.

TABLE 3

| | % | % | % |
|---|---|---|---|
| Sample | Carbon | Oxygen | Nitrogen |
| Poretics (0.1 m) | 83.8 | 15.4 | 0.4 |
| Poretics (0.1 m) + AApp | 84.1 | 14.9 | 0.5 |
| Poretics (0.1 m) + AApp + Collagen I + | 72.0 | 17.0 | 10.6 |
| NaCNBH3 | | | |
| Poretics (0.1 m) + AApp + Collagen IV + | 70.1 | 18.3 | 11.6 |
| NaCNBH3 | | | |
| Poretics (0.1 m) + AApp + Fibronectin + | 77.7 | 16.5 | 5.3 |
| NaCNBH3 | | | |

Table 3 shows that the attachment methodology is also effective when applied to porous substrates, in the present case track-etched membranes, and that other glycoproteins (collagen IV, fibronectin) can be immobilized with the method of the invention.

TABLE 4

| | % | % | % | % |
|---|---|---|---|---|
| Sample | Carbon | Oxygen | Nitrogen | Fluorine |
| Z-Por 1.1 | 32.0 | 23.0 | 1.9 | 43.1 |
| Z-Por + AApp | 54.2 | 21.1 | 0.6 | 24.1 |
| Z-Por + AApp + | 49.6 | 20.9 | 8.7 | 20.7 |
| Vitrogen + NaCNBH3 | | | | |

Table 4 shows that the method of the invention is applicable to and useful for bulk materials intended for ophthalmic applications (the example being Z-Por 1.1). The smaller nitrogen signal compared with the above tables does not indicate incomplete coverage by collagen; it is a function of the rough and porous substrate surface topology which reduces the relative emission intensity of the nitrogen signal in XPS from surface-attached molecules compared with the case when the sample is flat. Also, in this case the acetaldehyde plasma polymer interlayer was deposited to a thickness of less than 10 nm, as' is evident by the persistence of a (reduced) fluorine signal.

TABLE 5
Biological performance data: in vitro cell
attachment to acetaldehyde plasma
polymer on fluorinated co-ethylene propylene

| Surface | % Attachment | standard deviation |
|---|---|---|
| TCPS | 100.00 | 2.00 |
| FEP | 3.00 | 0.00 |
| AApp | 111.00 | 0.60 |
| AApp/collagen | 134.00 | 0.30 |

Assay relative to tissue culture polystyrene (TCPS), normalized to 100%.

Test methodology as described in J. G. Steele, G. Johnson, H. J. Griesser and P. A. Underwood, Biomaterials, 18, 1541 (1997).

1. A process for coupling an adhesive glycoprotein to a surface of a bulk material comprising the following steps:
a) a layer of a volatile aldehyde is deposited to the surface of the bulk material from a gas plasma atmosphere,
b) the layer so formed on the bulk material is contacted with a cell-adhesive glycoprotein having amino groups,
c) the coupling of the amino groups of the glycoprotein to the carbonyl groups of the deposited aldehyde layer is strenghthened by transforming the primarily formed —CH═N— bonds in the presence of a reducing agent into —CH₂—NH— bonds.

2. A process according to claim 1 wherein the aldehyde comprises up to 9 carbon atoms, preferably 2 to 4 carbon atoms.

3. A process according to claim 1 wherein the aldehyde is acetaldehyde.

4. A process according to claim 1 wherein the cell-adhesive glycoprotein is selected from collagen, fibronectin, vitronectin and laminin.

5. A process according to claim 1 wherein the cell-adhesive glycoprotein is collagen.

6. A cell growth material comprising a bulk material having at least one surface to which a cell adhesive glycoprotein having amino groups is coupled characterized in that said amino groups are covalently bonded to aldehyde groups of a layer of a volatile aldehyde, deposited to the surface of said bulk material from a gas plasma atmosphere, via first formed —CH═N— groups which have been transformed into —CH₂—NH— groups in the presence of a reducing agent.
